(19) United States
(12) Patent Application Publication
(10) Pub. No.: US 2008/0312268 A1
(43) Pub. Date: Dec. 18, 2008

(54) Substituted tricyclic piperidone compounds

(75) Inventors: Stefan Schunk, Aachen (DE); Stefan Oberboersch, Aachen (DE); Hagen-Heinrich Hennies, Simmerath (DE); Martin Maier, Tuebingen (DE); Anton Khartulyari, Tuebingen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GMBH, Aachen (DE)

(21) Appl. No.: 12/143,614

(22) Filed: Jun. 20, 2008

Related U.S. Application Data
(63) Continuation of application No. PCT/EP2006/012223, filed on Dec. 19, 2006.

(30) Foreign Application Priority Data
Dec. 22, 2005 (DE) 10 2005 061 426.4

(51) Int. Cl. A61K 31/439; C07D 221/22; A61P 25/32; A61P 25/30; A61P 25/00

(52) U.S. Cl. 514/295; 546/97

(57) ABSTRACT

Substituted tricyclic piperidone derivatives corresponding to Formula I:

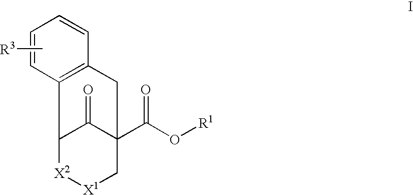

a method for producing such compounds; pharmaceutical compositions containing such compounds, and the use of such compounds to treat pain, depression, urinary incontinence, diarrhea, pruritus, alcohol and drug abuse, drug dependency, lethargy and/or anxiety.

### CROSS REFERENCE TO RELATED APPLICATIONS

This application is a continuation of international patent application no. PCT/EP2006/012223, filed Dec. 19, 2006, designating the United States of America, and published in German on Jul. 19, 2007 as WO 2007/079929, the entire disclosure of which is incorporated herein by reference. Priority is claimed based on Federal Republic of Germany patent application no. DE 10 2005 061 426.4, filed Dec. 22, 2005.

### BACKGROUND OF THE INVENTION

The present application relates to substituted tricyclic piperidone derivatives, methods for their production, medicaments containing these compounds and the use of substituted tricyclic piperidone derivatives for the production of medicaments.

A great deal of importance is attached by doctors to the treatment of chronic and non-chronic pain conditions. There is a worldwide need for highly effective pain therapies. The urgent need for action to find targeted, patient-appropriate treatment for chronic and non-chronic pain conditions, this being understood to be the successful and satisfactory treatment of pain for the patient, is reflected in the large number of scientific works that have been published in recent times in the field of applied analgesics and basic research into nociception.

Classic opioids such as morphine are very effective in the treatment of severe to very severe pain. Their use is restricted, however, by the known side-effects such as for example respiratory depression, vomiting, sedation, obstipation and tolerance development. Furthermore, they are less effective for neuropathic or incidental pain, which tumour patients in particular suffer from.

WO 9951602 discloses bridged cyclohexane rings substituted with a keto group, which influence the nicotinic acetylcholine receptor. However, these compounds neither carry a carboxylic acid ester nor have a fused phenyl ring.

### SUMMARY OF THE INVENTION

An object underlying the invention was to provide new analgesically active substances which are suitable for the treatment of pain—in particular also chronic and neuropathic pain.

The invention therefore provides substituted tricyclic piperidone derivatives corresponding to the formula I:

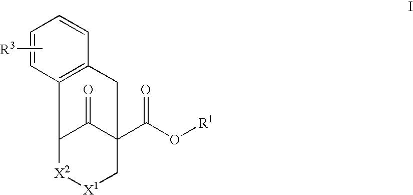

wherein
R¹ denotes methyl, ethyl or phenyl;
X₁ denotes NR² and X₂ for CH₂; or
X₂ denotes NR² and X₁ for CH₂;
R² denotes C₁₋₈ alkyl, saturated or unsaturated, branched or unbranched, mono- or polysubstituted or unsubstituted, optionally having a heteroatom as chain segment; aryl, C₃₋₈ cycloalkyl or heteroaryl bound via a saturated or unsaturated, branched or unbranched, substituted or unsubstituted C₁₋₈ alkyl group, optionally having a heteroatom as chain segment, in each case unsubstituted or mono- or polysubstituted; (C═O)R⁴, (C═O)NR⁵R⁶, (C═S)NR⁵R⁶, SO₂R⁷ or (C═O)OR⁸;
R³ denotes H, F, Cl, Br, OH, OCH₃, SCH₃, NO₂, CN; C₁₋₈ alkyl or C₃₋₈ cycloalkyl, in each case saturated or unsaturated, branched or unbranched, mono- or polysubstituted or unsubstituted; phenyl, benzyl or phenethyl, in each case unsubstituted or mono- or polysubstituted with F, Cl, OH, OCH₃, SCH₃, NO₂, CN, CF₃, methyl or ethyl;
R⁴, R⁷ and R⁸ denote C₁₋₈ alkyl or C₃₋₈ cycloalkyl, in each case saturated or unsaturated, branched or unbranched, mono- or polysubstituted or unsubstituted, optionally having an N, O, S heteroatom as chain segment; aryl, or heteroaryl, in each case mono- or polysubstituted or unsubstituted; aryl or C₃₋₈ cycloalkyl or heteroaryl bound via a saturated or unsaturated, branched or unbranched, substituted or unsubstituted C₁₋₈ alkyl group, optionally having an N, O, S heteroatom as chain segment, in each case unsubstituted or mono- or polysubstituted;
R⁵ and R⁶ mutually independently denote H; C₁₋₈ alkyl or C₃₋₈ cycloalkyl, in each case saturated or unsaturated, branched or unbranched, mono- or polysubstituted or unsubstituted, optionally having an N, O, S heteroatom as chain segment; aryl, or heteroaryl, in each case mono- or polysubstituted or unsubstituted; aryl or C₃₋₈ cycloalkyl or heteroaryl bound via a saturated or unsaturated, branched or unbranched, substituted or unsubstituted C₁₋₈ alkyl group, optionally having an N, O, S heteroatom as chain segment, in each case unsubstituted or mono- or polysubstituted; wherein R⁵ and R⁶ cannot simultaneously be H; or
R⁵ and R⁶ together form a five-, six- or seven-membered ring, which can be saturated or unsaturated but not aromatic, which optionally contains a further heteroatom from the group S, O or NR⁹ and which can be substituted with benzyl or C₁₋₅ alkyl,
   wherein R⁹ denotes C₁₋₅ alkyl, branched or unbranched; phenyl or benzyl, unsubstituted or mono- or polysubstituted with F, Cl, OH, OCH₃, SCH₃, NO₂, CN, CF₃, methyl or ethyl,
   in the form of the racemate; the enantiomers, diastereomers, mixtures of enantiomers or diastereomers or a single enantiomer or diastereomer; the bases and/or salts of physiologically compatible acids.

Within the meaning of this invention the expressions “C₁₋₃ alkyl”, “C₁₋₅ alkyl” and “C₁₋₈ alkyl” encompass acyclic saturated or unsaturated hydrocarbon radicals, which can be branched or straight-chained and unsubstituted or mono- or polysubstituted, having 1 to 3 C atoms or 1 to 5 C atoms or 1 to 8 C atoms, i.e. C₁₋₃ alkanyls, C₂₋₃ alkenyls and C₂₋₃ alkynyls or C₁₋₅ alkanyls, C₂₋₅ alkenyls and C₂₋₅ alkynyls or C₁₋₈ alkanyls, C₂₋₈ alkenyls and C₂₋₈ alkynyls. Alkenyls have at least one C—C double bond and alkynyls have at least one C—C triple bond. Alkyl is advantageously selected from the group encompassing methyl, ethyl, n-propyl, 2-propyl, n-butyl, isobutyl, sec-butyl, tert-butyl, n-pentyl, isopentyl, neopentyl, n-hexyl, 2-hexyl, ethylenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl, hexynyl, heptyl, heptenyl, heptynyl, octyl, octenyl and octynyl. Methyl, ethyl, n-propyl, n-butyl, sec-butyl and isobutyl are particularly advantageous. Methyl is most particularly advantageous.

If two substituents of an N atom “together form a five-, six- or seven-membered ring, which can be saturated or unsaturated but not aromatic, which optionally contains a further heteroatom from the group S, O or N”, within the meaning of this invention this means that the two substituents form a ring which encompasses the N atom. Rings from the group comprising pyrrolidine, piperidine, azepan, piperazine, diazepan, imidazolidine, morpholine, thiomorpholine, oxazepan, thiazepan, oxazole or thiazolidine are advantageous. Piperidine, piperazine, morpholine and thiomorpholine are particularly preferred.

Within the meaning of this invention, the expression “aryl” denotes aromatic hydrocarbons, inter alia phenyls and naphthyls. The aryl radicals can also be fused with other saturated, (partially) unsaturated or aromatic ring systems. Each aryl radical can be present in unsubstituted or mono- or polysubstituted form, wherein the aryl substituents can be the same or different and can be at any desired and possible position of the aryl. Aryl is advantageously selected from the group containing phenyl, 1-naphthyl, 2-naphthyl, each of which can be substituted or mono- or polysubstituted. The phenyl radical is particularly advantageous.

The expression “heteroaryl” denotes a 5-, 6- or 7-membered cyclic aromatic radical containing at least 1, optionally also 2, 3, 4 or 5 heteroatoms, wherein the heteroatoms can be the same or different and the heterocyclic compound can be unsubstituted or mono- or polysubstituted; if the heterocyclic compound is substituted, the substituents can be the same or different and can be at any desired and possible position of the heteroaryl. The heterocyclic compound can also be part of a bicyclic or polycyclic system. Preferred heteroatoms are nitrogen, oxygen and sulfur. It is preferable for the heteroaryl radical to be selected from the group including pyrrolyl, indolyl, furyl (furanyl), benzofuranyl, thienyl (thiophenyl), benzothienyl, benzothiadiazolyl, benzothiazolyl, benzotriazolyl, benzodioxolanyl, benzodioxanyl, phthalazinyl, pyrazolyl, imidazolyl, thiazolyl, oxazolyl, isoxazoyl, pyridyl, pyridazinyl, pyrimidinyl, pyrazinyl, pyranyl, indazolyl, purinyl, indolizinyl, quinolinyl, isoquinolinyl, quinazolinyl, carbazolyl, phenazinyl, phenothiazinyl or oxadiazolyl, wherein the bond to the compounds having the general structure I can be made via any desired and possible ring member of the heteroaryl radical. Pyridyl, furyl, thienyl and indolyl are particularly preferred.

For the purposes of the present invention the expression “aryl or heteroaryl bound via C₁₋₃ alkyl” means that C₁₋₃ alkyl and aryl or heteroaryl have the meanings defined above and the aryl or heteroaryl radical is bound to the compound having the general structure I via a C₁₋₃ alkyl group. Benzyl is particularly advantageous within the meaning of this invention.

In connection with “alkyl” or a “five-, six- or seven-membered ring, which can be saturated or unsaturated but not aromatic”, the term “substituted” within the meaning of this invention is understood to mean the substitution of a hydrogen radical with F, Cl, Br, I, —CN, NH₂, NH—C₁₋₆ alkyl, NH—C₁₋₆ alkyl-OH, C₁₋₆ alkyl, N(C₁₋₆ alkyl)₂, N(C₁₋₆ alkyl-OH)₂, NO₂, SH, S—C₁₋₆ alkyl, S-benzyl, O—C₁₋₆ alkyl, OH, O—C₁₋₆ alkyl-OH, ═O, O-benzyl, C(═O)C₁₋₆ alkyl, CO₂H, CO₂—C₁₋₆ alkyl or benzyl, wherein polysubstituted radicals are understood to be radicals which are substituted multiple times, for example twice or three times, at different or the same atoms, for example substituted three times at the same C atom, as in the case of CF₃ or —CH₂CF₃, or at different points, as in the case of —CH(OH)—CH═CH—CHCl₂. The multiple substitution can be performed with identical or different substituents. For the purposes of the present invention, “mono- or polysubstituted” in connection with alkyl or a saturated or unsaturated ring, which may not be aromatic, means benzyl or methyl.

With reference to “aryl” and “heteroaryl”, within the meaning of this invention “mono- or polysubstituted” is understood to mean the mono- or poly- (i.e. di-, tri- or tetra-)substitution of one or more hydrogen atoms of the ring system with F, Cl, Br, I, CN, NH₂, NH—C₁₋₆ alkyl, NH—C₁₋₆ alkyl-OH, N(C₁₋₆ alkyl)₂, N(C₁₋₆ alkyl-OH)₂, NO₂, SH, S—C₁₋₆ alkyl, OH, O—C₁₋₆ alkyl, O—C₁₋₆ alkyl-OH, C(═O)C₁₋₆ alkyl, CO₂H, CO₂—C₁₋₆ alkyl, CF₃, C₁₋₆ alkyl; at one or more optionally different atoms (wherein a substituent can optionally itself be substituted). The multiple substitution is performed with identical or with different substituents. Preferred substituents for “aryl” and “heteroaryl” are —F, —Cl, —CF₃, —O—CH₃, methyl, ethyl, n-propyl, nitro, tert-butyl and —CN. Methyl is particularly preferred.

Within the meaning of this invention, the concept of the salt formed with a physiologically compatible acid is understood to mean salts of the individual active ingredient with inorganic or organic acids which are physiologically—in particular for use in humans and/or mammals—compatible. Hydrochloride is particularly preferred. Examples of physiologically compatible acids are: hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, 1,1-dioxo-1,2-dihydro-1λ⁶-benzo[d]isothiazol-3-one (saccharinic acid), monomethyl sebacic acid, 5-oxo-proline, hexane-1-sulfonic acid, nicotinic acid, 2-, 3- or 4-aminobenzoic acid, 2,4,6-trimethylbenzoic acid, α-lipoic acid, acetylglycine, hippuric acid, phosphoric acid and/or aspartic acid. Hydrochloric acid is particularly preferred.

Substituted tricyclic piperidone derivatives having the general formula I are preferred in which X₁ denotes NR² and X₂ for CH₂.

Also preferred are tricyclic piperidone derivatives having the general formula I in which R² denotes C₁₋₅ alkyl, saturated or unsaturated, branched or unbranched; aryl, C₃₋₈ cycloalkyl or heteroaryl bound via a saturated or unsaturated, branched or unbranched, substituted or unsubstituted C₁₋₃ alkyl group, in each case unsubstituted or mono- or polysubstituted.

Tricyclic piperidone derivatives having the general formula I are particularly preferred in which R² denotes C₁₋₅ alkyl, branched or unbranched, unsubstituted and saturated; phenyl, naphthyl, pyridyl, furyl, thienyl and indolyl bound via a C₁₋₃ alkyl group, in each case unsubstituted or mono- or polysubstituted with —F, —Cl, —CF₃, —O—CH₃, methyl, ethyl, n-propyl, nitro, tert-butyl and —CN; in particular methyl or benzyl.

Additionally preferred are tricyclic piperidone derivatives having the general formula I in which R² denotes (C═O)R⁴, (C═O)NR⁵R⁶, (C═S)NR⁵R⁶, SO₂R⁷ or (C═O)OR⁸.

Also preferred are tricyclic piperidone derivatives having the general formula I, in which R⁴, R⁷ and R⁸ denote C₁₋₅ alkyl or C₃₋₈ cycloalkyl, branched or unbranched; aryl, or heteroaryl, in each case mono- or polysubstituted or unsubstituted; aryl, C₃₋₈ cycloalkyl or heteroaryl bound via a C₁₋₃ alkyl group, in each case unsubstituted or mono- or polysubstituted.

Particularly preferred are tricyclic piperidone derivatives having the general formula I, in which R⁴, R⁷ and R⁸ denote methyl, ethyl, n-propyl, isopropyl, n-butyl or tert-butyl; phenyl, naphthyl, thienyl, furyl or pyridyl, in each case mono- or polysubstituted with F, Cl, CF₃, NO₂, CH₃, OH, SH, OCH₃ or SCH₃ or unsubstituted; phenyl, naphthyl, thienyl, furyl or pyridyl bound via a C₁₋₃ alkyl group, in each case unsubstituted or mono- or polysubstituted with F, Cl, CF₃, NO₂, CH₃, OH, SH, OCH₃ or SCH₃; in particular methyl, ethyl, phenyl or tosyl.

Also preferred are tricyclic piperidone derivatives having the general formula I, in which R⁵ and R⁶ mutually independently denote H; C₁₋₅ alkyl or C₃₋₈ cycloalkyl, branched or unbranched; aryl, or heteroaryl, in each case mono- or polysubstituted or unsubstituted; aryl or C₃₋₈ cycloalkyl or heteroaryl bound via a C₁₋₃ alkyl group, in each case unsubstituted or mono- or polysubstituted; or

R⁵ and R⁶ together form piperidine, piperazine, morpholine or thiomorpholine.

Particularly preferred are tricyclic piperidone derivatives corresponding to formula I, in which R⁵ and R⁶ mutually independently denote H; methyl, ethyl, n-propyl, isopropyl, n-butyl or tert-butyl; phenyl, naphthyl, thienyl, furyl or pyridyl, in each case mono- or polysubstituted with F, Cl, CF₃, NO₂, CH₃, OH, SH, OCH₃ or SCH₃ or unsubstituted; phenyl, naphthyl, thienyl, furyl or pyridyl bound via a C₁₋₃ alkyl group, in each case unsubstituted or mono- or polysubstituted with F, Cl, CF₃, NO₂, CH₃, OH, SH, OCH₃ or SCH₃; in particular H or phenyl.

Also preferred are tricyclic piperidone derivatives having the general formula I, in which R³ denotes H, F, Cl, OH, OCH₃, SCH₃, NO₂, CN, CF₃, methyl, ethyl or benzyl, in particular for H, CN, OCH₃ or methyl.

Most particularly preferred are substituted tricyclic piperidone derivatives selected from the group consisting of:
Ethyl-N-benzyl-1-oxo-1,3,4,6-tetrahydro-1,5-methano-3-benzazocine-5(2H)-carboxylate;
Ethyl-N-benzyl-10-methyl-11-oxo-1,3,4,6-tetrahydro-1,5-methano-3-benzazocine-5(2H)-carboxylate;
Ethyl-N-benzyl-8-methoxy-11-oxo-1,3,4,6-tetrahydro-1,5-methano-3-benzazocine-5(2H)-carboxylate;
Ethyl-N-methyl-11-oxo-1,3,4,6-tetrahydro-1,5-methano-3-benzazocine-5(2H)-carboxylate hydrochloride;
Ethyl-N-10-dimethyl-11-oxo-1,3,4,6-tetrahydro-1,5-methano-3-benzazocine-5(2H)-carboxylate;
Diethyl-11-oxo-1,3,4,6-tetrahydro-1,5-methano-3-benzazocine-3,5(2H)-dicarboxylate;
Ethyl-N-(anilinocarbonyl)-11-oxo-1,3,4,6-tetrahydro-1,5-methano-3-benzazocine-5(2H)-carboxylate;
Ethyl-N-tosyl-11-oxo-1,3,4,6-tetrahydro-1,5-methano-3-benzazocine-5(2H)-carboxylate; and
Ethyl-N-benzyl-9-cyano-11-oxo-1,3,4,6-tetrahydro-1,5-methano-3-benzazocine-5(2H)-carboxylate.

The invention also provides a method for producing a tricyclic piperidone derivative according to the invention.

Tricyclic piperidone derivatives having the general formula I, in which X¹ denotes NR² and X² denotes CH₂, can be produced in accordance with the following synthesis scheme:

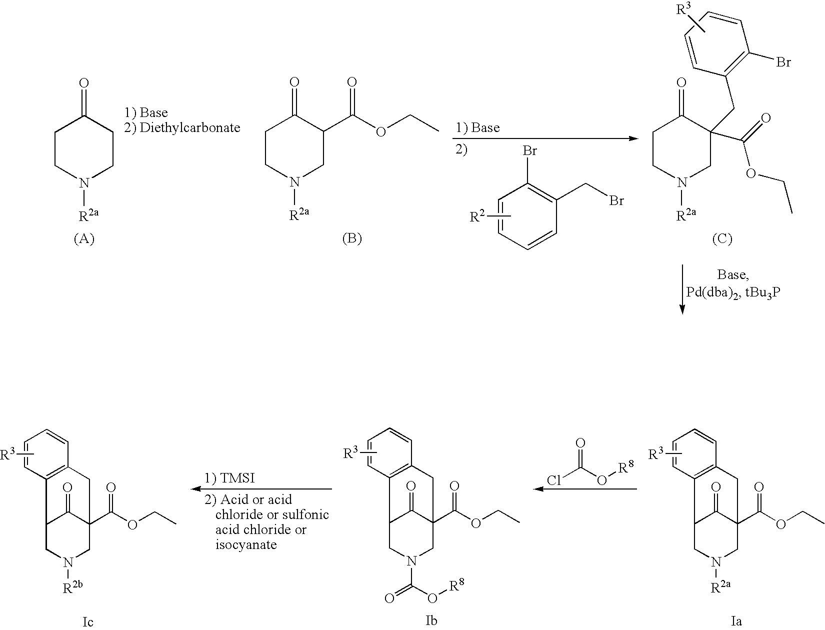

wherein
R^{2a} denotes C₁₋₈ alkyl, saturated or unsaturated, branched or unbranched, mono- or polysubstituted or unsubstituted, optionally having a heteroatom as chain segment; aryl, C₃₋₈ cycloalkyl or heteroaryl bound via a saturated or unsaturated, branched or unbranched, substituted or unsubstituted C₁₋₈ alkyl group, optionally having a heteroatom as chain segment, in each case unsubstituted or mono- or polysubstituted, and
R^{2b} denotes (C═O)R⁴, (C═O)NR⁵R⁶, (C═S)NR⁵R⁶, SO₂R⁷ or (C═O)OR⁸.

Piperidone derivatives having the general formula A are reacted here with diethyl carbonate, with addition of a base, for example diisopropylethylamine, triethylamine, pyridine, diethylamine, diisopropylamine, butyllithium, lithiumdiisopropylamide, sodium hydride, potassium hydride, sodium hydroxide, potassium hydroxide, sodium carbonate and potassium carbonate, in an organic solvent, for example acetone, dichloromethane, THF, diethylether, benzene, toluene. The products having the general formula B are reacted with the corresponding benzyl bromide in an organic solvent, for example acetone, acetonitrile, benzene, toluene, methanol, ethanol, dichloromethane, diethylether, tetrahydrofuran, with addition of a base, for example K₃PO₄, K₂CO₃, Na₂CO₃, NaOH, KOH, KOtBu, diisopropylethylamine, triethylamine, pyridine, diethylamine, diisopropylamine, at a temperature of 0 to 100° C. to form compounds having the general formula C. The compounds having the general formula C are reacted with a catalyst, such as for example Pd(dba)₂, PdCl₂, Pd(OAc)₂, and a corresponding ligand, for example t-Bu₃P, Ph₃P, in an organic solvent, for example acetone, benzene, toluene, acetonitrile, dichloromethane, diethylether, tetrahydrofuran, with addition of a base, for example K₃PO₄, K₂CO₃, Na₂CO₃, NaOH, KOH, diisopropylethylamine, triethylamine, pyridine, diethylamine, diisopropylamine, at a temperature of 40 to 130° C. under inert gas to form compounds having the general formula Ia. The compounds having the general formula Ia are dissolved in a compound having the general formula CIC(O)OR⁸, for example benzyloxycarbonylchloride, optionally dissolved in acetone, acetonitrile, benzene, dichloromethane, diethylether, toluene or tetrahydrofuran, and reacted at a temperature of 0 to 100° C. to form compounds having the general formula Ib. The products having the general formula Ib are reacted with trimethylsilyliodide in an organic solvent, for example acetone, acetonitrile, benzene, toluene, methanol, ethanol, dichloromethane, diethylether, tetrahydrofuran, at a temperature of −20 to 100° C. After extraction, the products are reacted with the corresponding electrophile, for example acid chlorides, acid anhydrides, sulfonic acid chlorides, isocyanates or isothiocyanates, in an organic solvent, for example acetone, benzene, toluene, acetonitrile, dichloromethane, diethylether, tetrahydrofuran, with addition of a base, for example K₃PO₄, K₂CO₃, Na₂CO₃, NaOH, KOH, diisopropylethylamine, triethylamine, pyridine, diethylamine, diisopropylamine, butyllithium, lithiumdiisopropylamide, sodium hydride, potassium hydride, at a temperature of −20° C. to 100° C. to form compounds having the general formula Ic.

Tricyclic piperidone derivatives having the general formula I, in which X¹ denotes CH₂ and X₂ denotes NR², can be produced according to the following synthesis scheme:

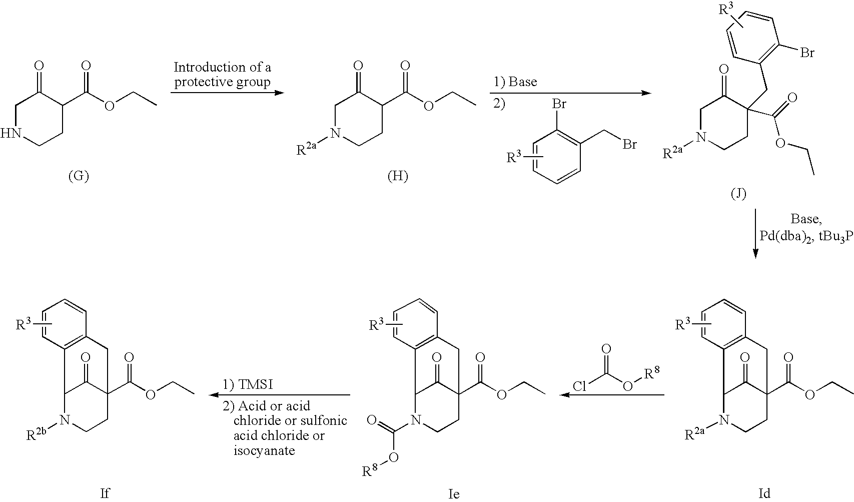

Compounds having the general formula H are reacted with the corresponding benzyl bromide in an organic solvent, for example acetone, acetonitrile, benzene, toluene, methanol, ethanol, dichloromethane, diethylether, tetrahydrofuran, with addition of a base, for example K₃PO₄, K₂CO₃, Na₂CO₃, NaOH, KOtBu, KOH, diisopropylethylamine, triethylamine, pyridine, diethylamine, diisopropylamine, at a temperature of 0 to 100° C. to form compounds having the general formula J. The compounds having the general formula J are reacted with a catalyst, such as for example Pd(dba)₂, PdCl₂, Pd(OAc)₂, and a corresponding ligand, for example t-Bu₃P, Ph₃P, in an organic solvent, for example acetone, benzene, toluene, acetonitrile, dichloromethane, diethylether, tetrahydrofuran, with addition of a base, for example K₃PO₄, K₂CO₃, Na₂CO₃, NaOH, KOH, diisopropylethylamine, triethylamine, pyridine, diethylamine, diisopropylamine, at a temperature of 40 to 130° C. under inert gas to form compounds having the general formula Id. The compounds having the general formula Id are dissolved in benzyloxycarbonylchloride, optionally dissolved in acetone, acetonitrile, benzene, dichloromethane, diethylether, toluene or tetrahydrofuran, and reacted at a temperature of 0 to 100° C. to form compounds having the general formula Ie. The products having the general formula Ie are reacted with trimethylsilyliodide in an organic solvent, for example acetone, acetonitrile, benzene, toluene, methanol, ethanol, dichloromethane, diethylether, tetrahydrofuran, at a temperature of −20 to 100° C. After extraction, the products are reacted with the corresponding electrophile, for example acid chlorides, acid anhydrides, sulfonic acid chlorides, isocyanates or isothiocyanates, in an organic solvent, for example acetone, benzene, toluene, acetonitrile, dichloromethane, diethylether, tetrahydrofuran, with addition of a base, for example K₃PO₄, K₂CO₃, Na₂CO₃, NaOH, KOH, diisopropylethylamine, triethylamine, pyridine, diethylamine, diisopropylamine, butyllithium, lithiumdiisopropylamide, sodium hydride, potassium hydride, at a temperature of −20° C. to 100° C. to form compounds having the general formula If.

The methods known to persons skilled in the art for producing benzyl bromides can be used in principle to prepare the necessary benzyl bromides. The benzyl bromides are thus prepared for example from the corresponding alcohols, esters or aldehydes by reduction and subsequent bromination. Friedel-Crafts or metallisation strategies can be used to prepare the aldehydes.

It has been shown that the substances according to the invention inhibit serotonin and noradrenalin re-uptake. Noradrenalin and serotonin re-uptake inhibitors have an antidepressive and anxiolytic effect, but are also suitable for the treatment of pain (Analgesics—from Chemistry and Pharmacology to Clinical Application, Wiley 2002, p. 265-284).

The substances according to the invention are suitable as pharmaceutical active ingredients in medicaments. The invention therefore also provides medicaments containing at least one substituted tricyclic piperidone derivative according to the invention, optionally along with suitable additives and/or auxiliary substances and/or optionally further active ingredients.

The medicaments according to the invention optionally contain, in addition to at least one substituted tricyclic piperidone derivative according to the invention, suitable additives and/or auxiliary substances, including carrier materials, fillers, solvents, diluents, dyes and/or binders, and can be administered as liquid dosage forms in the form of injection solutions, drops or juices, as semi-solid dosage forms in the form of granules, tablets, pellets, patches, capsules, plasters or aerosols. The choice of auxiliary substances, etc., and the amount thereof to use depend on whether the medicament is to be administered by oral, peroral, parenteral, intravenous, intraperitoneal, intradermal, intramuscular, intranasal, buccal, rectal or local means, for example on the skin, mucous membranes or in the eyes. Preparations in the form of tablets, pastilles, capsules, granules, drops, juices and syrups are suitable for oral administration; solutions, suspensions, easily reconstitutable dry preparations and sprays are suitable for parenteral, topical and inhalative administration. Tricyclic piperidone derivatives according to the invention in a depot formulation, in dissolved form or in a plaster, optionally with addition of agents promoting skin penetration, are suitable preparations for percutaneous administration. Preparation forms suitable for oral or percutaneous administration can deliver the tricyclic piperidone derivatives according to the invention on a delayed release basis. Other additional active ingredients known to the person skilled in the art can be added in principle to the medicaments according to the invention.

The amount of active ingredient to be administered to the patient varies according to the weight of the patient, the type of administration, the indication and the severity of the illness. 0.005 to 20 mg/kg, preferably 0.05 to 5 mg/kg, of at least one tricyclic piperidone derivative according to the invention are conventionally administered.

The medicament can contain a tricyclic piperidone derivative according to the invention as a pure diastereomer and/or enantiomer, as a racemate or as a non-equimolar or equimolar mixture of diastereomers and/or enantiomers.

The invention also provides the use of a tricyclic piperidone derivative according to the invention to produce a medicament for the treatment of pain, in particular of acute, neuropathic or chronic pain.

The invention also provides the use of a tricyclic piperidone derivative according to the invention to produce a medicament for the treatment of depression and/or for anxiolysis.

The substituted tricyclic piperidone derivatives having the general formula I are also suitable for the treatment of urinary incontinence, diarrhoea, pruritus, alcohol and drug abuse, drug dependency and avolition.

The invention therefore also provides the use of a substituted tricyclic piperidone derivative having the general formula I to produce a medicament for the treatment of urinary incontinence, diarrhoea, pruritus, alcohol and drug abuse, drug dependency and lethargy (avolition).

### EXAMPLES

### General Procedures

### GP 1-GP 4

### GP 1 Synthesis of Bromobenzyl-Substituted Oxopiperidine Derivatives

The corresponding benzyl bromide (1.2 eq.) dissolved in dry tetrahydrofuran was added to a suspension of the corresponding piperidine carboxylate (B or H; 1 eq.) and dry K₂CO₃ (3.9 eq.) in dry acetone under a nitrogen atmosphere. The reaction mixture was refluxed for 6 h. The inorganic salts were then filtered off and washed with acetone. The combined organic phases were then concentrated to small volume and purified by column chromatography. The desired bromobenzyl-substituted oxopiperidine derivatives C and J were obtained in this way.

### GP 2 Synthesis of Benzazocine Carboxylates

The corresponding bromobenzyl-substituted oxopiperidine derivative C or J (1 eq.), K₃PO₄ (2 eq.) and Pd(dba)₂ (2 mol %) were placed in a dry Schlenk flask flooded with argon. Toluene and t-Bu₃P (4 mol %) were added to this mixture under an argon atmosphere and stirred in an oil bath at 110° C. for 12 hours. The reaction mixture was then cooled to room temperature, diluted with diethylether, filtered through Celite and concentrated to small volume under vacuum. The crude product was purified by column chromatography and the desired benzazocine carboxylates Ia and Id were obtained in this way.

GP 3 Synthesis of cbz-Protected Benzazocine Carboxylates

The corresponding benzyl-protected benzazocine carboxylates D and J (1 eq.) were dissolved in benzyloxycarbonyl chloride and heated at 80° C. until completion of the reaction. The solvent was then removed by vacuum distillation. The crude product was purified by column chromatography and the desired cbz-benzazocine carboxylates Ib and Ie were obtained in this way.

### GP 4 Elimination of the cbz Group

A solution of the corresponding cbz-protected benzazocine carboxylate Ib or Ie (1 eq.) in acetonitrile was kept at a constant temperature in a water bath and trimethylsilyliodide (8.1 eq.) was added dropwise. After the reaction mixture had been stirred for 1 hour at room temperature, the reaction mixture was concentrated to small volume under vacuum. Water was added to the residue obtained and the mixture was washed with dichloromethane. Concentration of the aqueous phase to small volume led to the desired products.

### GP 5 Synthesis of Bromobenzyl-Substituted Oxopiperidine Derivatives

The corresponding piperidine carboxylate (B or H; 1 eq.) is added to a suspension of sodium hydride (1.05 eq.) in toluene. The reaction mixture was heated for 1 h at 80° C. The corresponding bromobenzyl dimethyl anilinium salt was then added and the reaction mixture was refluxed for 6 hours. After cooling, the reaction mixture was carefully poured onto water, the organic phase separated off, washed with saturated NaCl solution and dried with sodium sulfate. The combined organic phases were then concentrated to small volume and purified by column chromatography. The desired bromobenzyl-substituted oxopiperidine derivatives C and J were obtained in this way.

### GP 6 Synthesis of N-Carbamatobenzazocine Carboxylates

Triethylamine (1.6 eq.) was added to a solution of the salt obtained according to GP 4 (1 eq.) in acetonitrile and the mixture was stirred at room temperature for 1 hour. The corresponding isocyanate (1.6 eq.) was then added and the reaction mixture was stirred at room temperature for 24 hours. The solvent was then removed under vacuum. The crude product was purified by column chromatography and the desired N-carbamatobenzazocine carboxylates Ic and If were obtained in this way.

### GP 7 Synthesis of N-Sulfonylated Benzazocine Carboxylates

Triethylamine (1.6 eq.) was added to a solution of the salt obtained according to GP 4 (1 eq.) in acetonitrile and the mixture was stirred at room temperature for 1 hour. The corresponding sulfonyl chloride (1.6 eq.) was then added and the reaction mixture was stirred at room temperature for 24 hours. The solvent was then removed under vacuum. The crude product was purified by column chromatography and the desired N-sulfonylated benzazocine carboxylates Ic and If were obtained in this way.

### Ethyl-3-(2-bromobenzyl)-N-benzyl-4-oxopiperidine-3-carboxylate (1)

In accordance with GP 1, ethyl-1-benzyl-4-oxopiperidine-3-carboxylate (3.93 g, 15 mmol) dissolved in 25 ml acetone was reacted with 2-bromobenzyl bromide (4.5 g, 18.0 mmol) dissolved in 25 ml THF. After purification by column chromatography (silica gel, Et₂O/EtOAc, 20:1), the desired product was present in the form of a pale yellow oil. Yield: 4.72 g (73%)

¹H NMR (400 MHz, CDCl₃): δ=7.43 (d, J=8.0 Hz, 1H, aromatic), 7.25-7.07 (m, 7H, aromatic), 7.00-6.94 (m, 1H (aromatic)), 4.10-3.95 (m, 2H), 3.57 (d, J=13.3 Hz, 2H), 3.51 (dd, J=11.6 Hz, J=2.9 Hz, 1H), 3.43 (d, J=5.9 Hz, 1H), 3.39 (d, J=7.1 Hz, 1H), 3.10 (d, J=14.5 Hz, 1H), 2.97-2.89 (m, 1H), 2.86-2.76 (m, 1H), 2.37-2.30 (m, 1H), 2.26 (d, J=11.6 Hz, 1H), 2.25-2.17 (m, 1H), 1.03 (t, J=7.1 Hz, CH₃); ¹³C NMR (100 MHz, CDCl₃): δ=205.25 (carbonyl), 170.72 (carbethoxy); 137.7, 136.3, 132.7, 132.1, 128.8, 128.2, 128.2, 127.2, 127.0, 125.9 (aromatic), 62.2, 61.8, 61.4, 61.0, 52.6, 40.5, 35.8, 13.8; HRMS (ESI): calculated for C₂₂H₂₄BrNO₃ [M+H]⁺: 430.10123, found 430.10129.

### Ethyl-3-(2-bromo-3-methylbenzyl)-N-benzyl-4-oxopiperidine-3-carboxylate (2)

In accordance with GP 1, ethyl-1-benzyl-4-oxopiperidine-3-carboxylate (1.31 g, 5.0 mmol) was reacted with 2-bromo-1-(bromomethyl)-3-methylbenzene (1.32 g, 5.0 mmol). After purification by column chromatography (silica gel, Et₂O/EtOAc, 20:1), the desired product was present in the form of colourless crystals. Yield: 1.2 g (54%). Melting point: 60-61° C.

¹H NMR (400 MHz, CDCl₃): δ=7.26-7.25 (m, 5H), 7.05-6.93 (m, 2H), 4.12-3.97 (m, 2H), 3.60 (d, J=13.1 Hz, 1H), 3.52 (dd, J=11.5, 3.0 Hz, 1H), 3.49 (d, J=14.4 Hz, 1H), 3.43 (d, J=13.1 Hz, 1H), 3.16 (d, J=14.4 Hz, 1H), 2.98-2.91 (m, 1H), 2.87-2.78 (m, 1H), 2.38-2.32 (m, 4H), 2.30 (d, J=11.6 Hz, 1H), 2.27-2.20 (m, 1H), 1.05 (t, J=7.1 Hz, 3H); ¹³C NMR (100 MHz, CDCl₃): δ=205.3, 170.8, 138.5, 137.8, 136.7, 129.4, 129.2, 128.9, 128.7, 128.2, 127.2, 126.3, 62.2, 61.8, 61.46, 61.1, 52.65, 40.5, 36.5, 24.5, 13.8; HRMS (ESI): calculated for C₂₃H₂₆BrNO₃ [M+H]⁺: 444.11688, found 444.11739.

### Ethyl-3-(2-bromo-5-methoxybenzyl)-N-benzyl-4-oxopiperidine-3-carboxylate (3)

In accordance with GP 1, ethyl-1-benzyl-4-oxopiperidine-3-carboxylate (2.61 g, 10.0 mmol) was reacted with 1-bromo-2-(bromomethyl)-4-methoxybenzene (2.95 g, 11.0 mmol). After purification by column chromatography (silica gel, Et₂O/EtOAc, 20:1), the desired product was present in the form of a yellow oil. Yield: 2.4 g (52%).

¹H NMR (400 MHz, CDCl₃): δ=7.34 (d, J=8.9 Hz, 1H), 7.27-7.18 (m, 5H), 6.74 (d, J=3.0 Hz, 1H), 6.59 (dd, J=8.9, 3.0 Hz, 1H), 4.15-4.00 (m, 2H), 3.69 (s, 3H), 3.63-3.54 (m, 2H), 3.47 (d, J=13.3 Hz, 1H), 3.37 (d, J=14.4 Hz, 1H), 3.10 (d, J=14.4 Hz, 1H), 3.00-2.93 (m, 1H), 2.89-2.80 (m, 1H), 2.50-2.33 (m, 1H), 2.30-2.21 (m, 2H), 1.09 (t, J=7.1 Hz, 3H); ¹³C NMR (100 MHz, CDCl₃): δ=205.3, 170.8, 158.5, 137.7, 137.2, 133.1, 128.9, 128.2, 127.2, 117.5, 116.4, 114.3, 62.3, 61.8, 61.4, 60.9, 55.4, 52.6, 40.5, 36.0 13.8; HRMS (ESI): calculated for C₂₃H₂₄BrNO₄ [M+H]⁺: 460.11180, found 460.11252.

### Ethyl-3-(2-bromobenzyl)-N-methyl-4-oxopiperidine-3-carboxylate (4)

In accordance with GP 5, NaH (235 mg, 5.85 mmol, 60% in mineral oil) and ethyl-1-methyl-4-oxopiperidine-3-carboxylate (1.07 g, 5.8 mmol) were reacted with o-bromobenzyl dimethyl anilinium bromide (2.06 g, 5.55 mmol). After purification by column chromatography (silica gel, petroleum ether/EtOAc, 1:1), the desired product was present in the form of a pale yellow oil. Yield: 550 mg (28%).

¹H NMR (400 MHz, CDCl₃): δ=7.50 (d, J=7.9 Hz, 1H), 7.21-7.16 (m, 2H), 7.09-7.02 (m, 1H), 4.17-4.07 (m, 2H), 3.46 (d, J=14.4 Hz, 1H), 3.43 (dd, J=11.6, 2.8 Hz, 1H), 3.17 (d, J=14.4 Hz, 1H), 3.05-2.96 (m, 1H), 2.94-2.84 (m, 1H), 2.97-2.89 (m, 1H), 2.45-2.39 (m, 1H), 2.33-2.24 (m, 4H), 2.20 (d, J=11.6 Hz, 1H), 2.25-2.17 (m, 1H), 1.04 (t, J=7.1 Hz, 3H); ¹³C NMR (100 MHz, CDCl₃): δ 204.9, 170.3, 136.1, 132.9, 132.2, 128.4, 127.1, 125.9, 62.9, 61.6, 61.6, 55.8, 45.6, 406, 36.0, 17.8; HRMS (ESI): calculated for C₁₆H₂₀BrNO₃ [M+H]⁺: 354.06993, found 354.07013.

### Ethyl-3-(2-bromo-3-methylbenzyl)-N-methyl-4-oxopiperidine-3-carboxylate (5)

In accordance with GP 5, ethyl-1-methyl-4-oxopiperidine-3-carboxylate (1.07 g, 5.8 mmol) was reacted with o-bromo-m-methylbenzyl dimethyl anilinium bromide (2.22 g, 5.7 mmol). After purification by column chromatography (silica gel, petroleum ether/Et₂₀, 1:1), the desired product was present in the form of a pale yellow oil. Yield: 410 mg (25%).

¹H NMR (400 MHz, CDCl₃): δ=7.10-7.05 (m, 2H), 7.00-6.96 (m, 1H), 4.19-4.06 (m, 2H), 3.53 (d, J=14.4 Hz, 1H), 3.43 (dd, J=11.6, 2.8 Hz, 1H), 3.22 (d, J=14.4 Hz, 1H), 3.04-2.96 (m, 1H), 2.94-2.84 (m, 1H), 2.45-2.39 (m, 1H), 2.40 (s, 3H), 2.32-2.25 (m, 1H), 2.30 (s, 3H), 1.15 (t, J=7.1 Hz, 3H); ¹³C NMR (100 MHz, CDCl₃): δ=204.9, 170.4, 138.6, 136.5, 129.5, 129.3, 128.6, 126.4, 62.9, 61.6, 61.5, 55.8, 45.6, 40.6, 36.7, 24.6, 13.8; HRMS (ESI): calculated for C₁₇H₂₂BrNO₃ [M+H]⁺: 368.08558, found 368.08568.

### Ethyl-N-benzyl-11-oxo-1,3,4,6-tetrahydro-1,5-methano-3-benzazocine-5(2H)-carboxylate (6)

In accordance with GP 2, ethyl-1-benzyl-3-(2-bromobenzyl)-4-oxopiperidine-3-carboxylate (1) (430 mg, 1 mmol) was reacted with K₃PO₄ (425 mg, 2 mmol), Pd(dba)₂ (11.5 mg, 2 mol %) and tBu₃P (1 ml of a 0.04 N solution in toluene, 4 mol %). After purification by column chromatography (silica gel, petroleum ether/Et₂O, 9:1), the desired product was present in the form of a yellow solid. Yield: 230 mg (65%), m.p. 117-119° C.

¹H NMR (400 MHz, CDCl₃): δ=7.31-7.13 (m, 6H, aromatic), 6.95-6.87 (m, 3H, aromatic), 4.25 (q, J=14.2, 7.1 Hz, CH₂ (carbethoxy)), 4.00 (d, J=17.2 Hz, 1H), 3.61-3.51 (m, 2H), 3.43 (d, J=17.2 Hz, 1H), 3.45-3.42 (m, 1H), 3.18 (dd, J=11.2, 2.8 Hz, 1H), 3.12 (d, J=11.2 Hz, 1H), 3.01 (ddd, J=10.6, 2.8, 2.8 Hz, 1H), 2.75 (dd, J=10.6, 2.4 Hz, 1H), 1.30 (t, J=7.1 Hz, CH₃); ¹³C NMR (100 MHz, CDCl₃): δ=207.4 (carbonyl), 170.2 (carbethoxy), 138.2, 137.3, 136.3, 128.2, 128.1, 127.5, 127.1, 127.0, 126.2, 126.2 (aromatic), 63.5, 61.9, 61.5, 60.2, 59.1, 52.7, 41.0, 14.1; HRMS (ESI): calculated for C₂₂H₂₃NO₃ [M+H]⁺: 350.17507, found 350.17524; methanol adduct (hemiacetal) calculated for C₂₃H₂₇NO₄ [M+CH₃OH+H]⁺: 382.20128, found 382.20076.

### Ethyl-N-benzyl-10-methyl-11-oxo-1,3,4,6-tetrahydro-1,5-methano-3-benzazocine-5(2H)-carboxylate (7)

Ethyl-3-(2-bromo-3-methylbenzyl)-N-benzyl-4-oxopiperidine-3-carboxylate (2) (455 mg, 1.02 mmol) was reacted in accordance with GP 2. After purification by column chromatography (silica gel, petroleum ether/EtOAc, 20:1), the desired product was present in the form of a colourless oil. Yield: 95 mg (30%).

¹H NMR (400 MHz, CDCl₃): δ=7.26-7.17 (m, 5H), 7.05-6.99 (m, 1H), 6.94-6.88 (m, 2H), 4.04 (q, J=14.2, 7.1 Hz, 2H), 3.51 (d, J=13.4 Hz, 1H), 3.36 (d, J=13.4 Hz, 1H), 3.07 (s, 2H), 2.70 (d, J=11.6 Hz, 1H), 2.55-2.47 (m, 1H), 2.41 (s, 3H), 2.37-2.27 (m, 1H), 2.25-2.18 (m, 1H), 2.14-2.05 (m, 1H), 1.11 (t, J=7.1 Hz, 3H); ¹³C NMR (100 MHz, CDCl₃): δ=205.3, 176.5, 138.8, 134.3, 129.7, 128.9, 128.6, 128.2, 128.2, 127.7, 126.9, 122.6, 62.2, 60.9, 57.8, 49.7, 40.5, 37.1, 35.0, 18.5, 13.9; HRMS (ESI): calculated for C₂₃H₂₅NO₃ [M+H]⁺: 364.19072, found 364.19171.

### Ethyl-N-benzyl-8-methoxy-11-oxo-1,3,4,6-tetrahydro-1,5-methano-3-benzazocine-5(2H)-carboxylate (8)

Ethyl-3-(2-bromo-5-methoxybenzyl)-N-benzyl-4-oxopiperidine-3-carboxylate (3) (577 mg, 1.25 mmol) was reacted in accordance with GP 2. After purification by column chromatography (silica gel, petroleum ether/EtOAc, 20:1), the desired product was present in the form of yellow crystals. Yield: 144 mg (40%), m.p. 84-85° C.

¹H NMR (400 MHz, CDCl₃): δ=7.19-7.15 (m, 3H), 6.97-6.93 (m, 2H), 6.86-6.82 (m, 1H), 6.75-6.72 (m, 2H), 4.27-4.20 (m, 2H), 3.97 (d, J=17.4 Hz, 1H), 3.84-3.82 (s, 3H), 3.56 (s, 2H), 3.42-3.35 (m, 2H), 3.15 (dd, J=11.2, 2.8 Hz, 1H), 3.08 (d, J=11.2, 1H), 2.98 (ddd, J=10.6, 2.8, 1H), 2.72 (dd, J=10.6, 2.5 Hz, 1H), 1.29 (t, J=7.1 Hz, 3H); ¹³C NMR (100 MHz, CDCl₃): δ=207.5, 170.3, 158.8, 138.2, 137.4, 129.6, 128.5, 128.2, 128.1, 127.1, 112.3, 111.4, 63.4, 62.0, 61.5, 60.3, 58.8, 55.4, 52.0, 41.1, 14.1; HRMS (ESI): calculated for C₂₃H₂₅NO₄ [M+H]⁺: 380.18563, found 380.18573; methanol adduct (hemiacetal) calculated for C₂₄H₂₉NO₅ [M+CH₃OH+H]⁺: 412.21185, found 412.21114.

### Ethyl-N-methyl-11-oxo-1,3,4,6-tetrahydro-1,5-methano-3-benzazocine-5(2H)-carboxylate hydrochloride (9)

In accordance with GP 2,1-methyl-3-(2-bromobenzyl)-4-oxopiperidine-3-carboxylate (4) (745 mg, 2.16 mmol) was reacted with K₃PO₄ (1.3 g, 6.1 mmol), Pd(dba)₂ (25 mg, 2 mol %) and tBu₃P (2 ml of a 0.04N solution in toluene, 4 mol %) and heated for 72 h in an oil bath at 120° C. After acid aqueous extraction, the desired product was present in the form of a yellow solid. Yield: 192 mg (25%).

¹H NMR (400 MHz, CDCl₃): δ=7.25-7.12 (m, 3H), 6.91 (d, J=7.5 Hz, 1H), 4.27 (q, J=14.3, 7.1 Hz), 4.02 (d, J=17.3 Hz, 1H), 3.52 (d, J=17.3 Hz, 1H), 3.39 (t, J=2.5 Hz, 1H), 3.15 (dd, J=11.3, 2.8, 1H), 3.04-2.96 (m, 2H), 2.67 (dd, J=10.7, 2.8, 1H), 2.25 (s, 3H), 1.31 (t, J=7.1 Hz, 3H); ¹³C NMR (100 MHz, CDCl₃): δ=207.5, 170.29, 137.3, 135.8, 127.5, 127.2, 126.6, 126.3, 66.6, 64.7, 61.6, 58.6, 52.5, 45.0, 41.0, 14.1; HRMS (ESI): calculated for C₁₆H₁₉NO₃ [M+H]⁺: 274.14377, found 274.14323; methanol adduct (hemiacetal) calculated for C₁₇H₂₃NO₄ [M+CH₃OH+H]⁺: 306.16998, found 306.16986.

### Ethyl-N-10-dimethyl-11-oxo-1,3,4,6-tetrahydro-1,5-methano-3-benzazocine-5(2H)-carboxylate (10)

Ethyl-3-(2-bromo-3-methylbenzyl)-N-methyl-4-oxopiperidine-3-carboxylate (5) (147 mg, 0.40 mmol) was reacted in accordance with GP 2 and heated for 72 h in an oil bath at 120° C. After purification by column chromatography (silica gel, petroleum ether/EtOAc, 20:1), the desired product was present in the form of crystals. Yield: 27 mg (25%).

¹H NMR (400 MHz, CDCl₃): δ=7.13-7.09 (m, 1H), 7.03-6.98 (m, 2H), 4.30-4.24 (m, 2H), 4.01 (d, J=17.3 Hz, 1H), 3.60 (t, J=2.5 Hz, 1H), 3.49 (d, J=17.3 Hz, 1H), 3.19 (dd, J=11.3, 2.8 Hz, 1H), 3.04-2.96 (m, 1H), 2.63 (dd, J=10.8, 3.0 Hz, 1H), 2.27 (s, 3H), 2.30 (s, 3H), 1.31 (t, J=7.1 Hz, 3H); ¹³C NMR (100 MHz, CDCl₃): δ=208.1, 170.4, 135.7, 135.3, 134.7, 128.0, 126.8, 124.6, 66.3, 62.4, 61.6, 58.5, 48.7, 45.1, 41.0, 18.5, 14.1.

### Diethyl-11-oxo-1,3,4,6-tetrahydro-1,5-methano-3-benzazocine-3,5(2H)-dicarboxylate (11)

Ethyl-N-benzyl-11-oxo-1,3,4,6-tetrahydro-1,5-methano-3-benzazocine-5(2H)-carboxylate (6) (225 mg, 0.64 mmol) was dissolved in ethoxycarbonyl chloride (5 ml) and heated for 3 days at 60° C. The excess of ethoxycarbonyl chloride was removed by vacuum distillation. After purification by column chromatography (silica gel, petroleum ether/EtOAc, 5:1), the desired product was present in the form of a colourless oil. Yield: 135 mg (64%). Conversion: approx. 75%

¹H NMR (400 MHz, CDCl₃): δ=7.23-7.14 (m, 2H), 7.13-7.00 (m, 2H), 4.79 (dd, J=13.6, 3.3 Hz, 0.6H), 4.60-4.37 (m, 0.5H), 4.32-4.22 (m, 2.6H), 4.05-3.87 (m, 1.6H), 3.77-3.51 (m, 4H), 3.47-3.30 (m, 1.3H), 1.31 (t, J=7.2 Hz, 3H), 1.13 (t, J=7.2 Hz, 1H), 0.77 (t, J=7.2 Hz, 2H); ¹³C NMR (100 MHz, CDCl₃): δ=205.3, 169.3, 155.8, 134.8, 134.4, 128.2, 127.7, 127.3, 126.8, 61.8, 61.6, 59.7, 53.9, 53.2, 52.5, 39.0, 14.1. According to the NMR spectra, (11) is a mixture of rotamers (˜2:1); HRMS (ESI): calculated for C₁₈H₂₁NO₅ [M+H]⁺: 332.14925, found 332.14910.

### 3-Benzyl-5-ethyl-11-oxo-1,3,4,6-tetrahydro-1,5-methano-3-benzazocine-3,5(2H)-dicarboxylate (12)

Ethyl-N-benzyl-11-oxo-1,3,4,6-tetrahydro-1,5-methano-3-benzazocine-5(2H)-carboxylate (6) (1.0 g, 2.8 mmol) was reacted in accordance with GP 3. After purification by column chromatography (silica gel, petroleum ether/EtOAc, 95:5), the desired product was present in the form of a colourless oil. Yield: 780 mg (70%).

¹H NMR (400 MHz, CDCl₃): δ 7.36-7-03 (m, 7H), 7.02-6.85 (m, 2H), 4.98-4.72 (m, 2H), 4.62-4.39 (m, 1.5H), 4.32-4.22 (m, 2.5H), 4.03-3.90 (m, 1H), 3.77-3.63 (m, 1H), 3.59-3.52 (m, 1.5H), 3.46-3.28 (m, 1.5H), 1.35-1.27 (m, 3H); ¹³C NMR (100 MHz, CDCl₃): δ 205.2, 169.3, 169.2, 155.6, 154.9, 136.0, 135.8, 134.8, 134.6, 134.3, 133.5, 128.5, 128.4, 128.4, 128.3, 128.2, 127.9, 127.8, 127.6, 127.4, 127.2, 126.9, 126.8, 67.6, 67.5, 61.9, 59.6, 59.1, 54.1, 53.9, 53.2, 52.4, 52.4, 39.2, 39.0, 14.1. According to the NMR spectra, (12) is a mixture of rotamers (˜2:1); HRMS (ESI): calculated for C₂₃H₂₃NO₅ [M+H]⁺: 394.16490, found 394.16512.

### Ethyl-11-oxo-1,3,4,6-tetrahydro-1,5-methano-3-benzazocine-5(2H)-carboxylate hydroiodide hydrate (13)

3-Benzyl-5-ethyl-11-oxo-1,3,4,6-tetrahydro-1,5-methano-3-benzazocine-3,5(2H)-dicarboxylate (12) (350 mg, 0.9 mmol) was reacted in accordance with GP 4. The desired product was obtained in the form of yellow crystals. Yield: 180 mg (50%) (×HI, ×H₂O) (×HI, ×H₂O). m.p. 170-172° C.

¹H NMR (400 MHz, CDCl₃): δ=9.30-9.10 (broad, 1H), 7.50-7.30 (broad, 1H), 7.30-7.17 (m, 4H), 4.25-4.13 (m, 2H), 3.64 (d, J=17.7 Hz, 1H), 3.53-3.44 (m, 1H), 3.43-3.31 (m, 2H), 3.17-3.09 (m, 2H), 3.06 (d, J=17.7 Hz, 1H), 1.25 (t, J=7.1 Hz, 3H); ¹³C NMR (100 MHz, CDCl₃): δ=171.0, 134.7, 134.3, 129.1, 127.9, 127.0, 126.3, 91.5, 61.4, 48.5, 48.3, 47.0, 44.2, 35.7, 13.9; HRMS (ESI): calculated for C₁₅H₁₈NO₃ [M]⁺: 260.12812, found 260.12857; methanol adduct (hemiacetal) calculated for C₁₆H₂₂NO₄ [M+CH₃OH]⁺: 292.15433, found 292.15432.

### Ethyl-N-(anilinocarbonyl)-11-oxo-1,3,4,6-tetrahydro-1,5-methano-3-benzazocine-5(2H)-carboxylate (14)

Ethyl-11-oxo-1,3,4,6-tetrahydro-1,5-methano-3-benzazocine-5(2H)-carboxylate hydroiodide hydrate (13) (45 mg, 0.11 mmol) was reacted with phenylisocyanate (20 μl, 0.18 mmol) in accordance with GP 6. The desired product was obtained in the form of pale yellow crystals. Yield: 35 mg (84%), m.p. 199-203° C.

¹H NMR (400 MHz, CDCl₃): δ=7.24-7.18 (m, 2H), 7.14-7.06 (m, 4H), 6.93-6.88 (m, 1H), 6.72 (d, J=7.8 Hz, 2H), 5.40-5.34 (broad, 1H), 4.86 (dd, J=13.6, 3.5 Hz, 1H), 4.29-4.21 (m, 2H), 3.98-3.87 (m, 2H), 3.66-3.59 (m, 3H), 3.49 (d, J=17.9 Hz, 1H), 1.27 (t, J=7.1 Hz, 3H); ¹³C NMR (100 MHz, CDCl₃): δ=204.6, 169.2, 155.1, 138.1, 135.1, 134.9, 128.6, 128.5, 128.1, 127.4, 127.3, 123.4, 120.5, 62.0, 59.8, 54.6, 53.5, 52.4, 39.0, 14.1; HRMS (ESI): calculated for C₂₂H₂₂N₂O₄ [M+H]⁺: 379.16523, found 379.16559; [M+Na]⁺: 401.14718, found 401.14749.

### Ethyl-N-tosyl-11-oxo-1,3,4,6-tetrahydro-1,5-methano-3-benzazocine-5(2H)-carboxylate (15)

Ethyl-11-oxo-1,3,4,6-tetrahydro-1,5-methano-3-benzazocine-5(2H)-carboxylate hydroiodide hydrate (13) (48 mg, 0.12 mmol) was reacted with toluenesulfonyl chloride (35 mg, 0.18 mmol) in accordance with GP 7. After purification by column chromatography (silica gel, petroleum ether/EtOAc, 3:1), the desired product was present in the form of a yellowish oil. Yield: 42 mg (80%).

¹H NMR (400 MHz, CDCl₃): δ=7.46 (d, J=8.3 Hz, 2H), 7.26-7.16 (m, 4H), 7.11 (d, J=7.6 Hz, 1H), 6.98 (d, J=7.6 Hz, 1H), 4.27 (dd, J=14.2, 7.1 Hz, 2H), 4.13 (dd, J=12.1, 3.3 Hz, 1H), 4.03 (d, J=17.5 Hz, 1H), 3.96-3.90 (m, 1H), 3.52-3.49 (m, 1H), 3.46 (d, J=17.6 Hz, 1H), 3.40 (d, J=12.1 Hz, 1H), 3.12 (dd, J=11.4, 2.3 Hz, 1H), 2.40 (s, 3H), 1.29 (t, J=7.1 Hz, 3H); ¹³C NMR (100 MHz, CDCl₃): δ=205.2, 169.0, 143.9, 134.8, 134.7, 134.2, 129.8, 128.0, 127.9, 127.2, 127.2, 127.1, 62.1, 58.1, 55.9, 54.9, 51.3, 34.0, 21.5, 14.1; HRMS (ESI): calculated for C₂₂H₂₃NO₅S [M+H]⁺: 414.13697, found 414.13655; [M+Na]⁺: 436.11738, found 436.11738.

### Ethyl-N-benzyl-9-cyano-11-oxo-1,3,4,6-tetrahydro-1,5-methano-3-benzazocine-5(2H)-carboxylate (16)

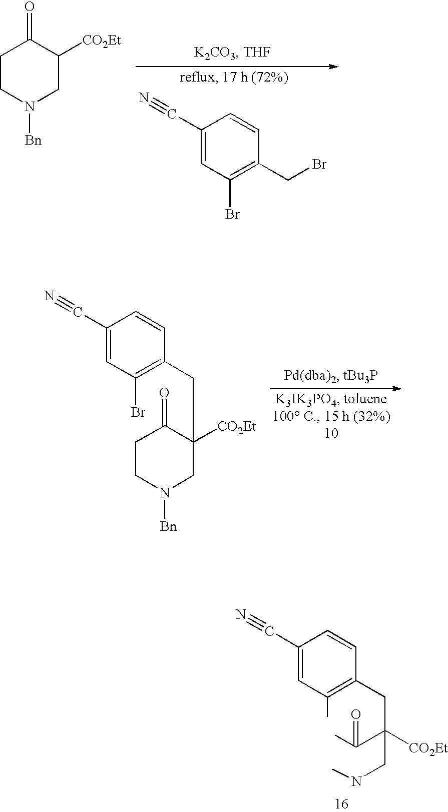

### Ethyl-3-(2-bromo-4-cyanobenzyl)-N-benzyl-4-oxopiperidine-3-carboxylate

A solution of 2-bromo-4-cyanobenzyl bromide (0.50 g, 1.8 mmol) in absolute THF (5 ml) was added under an inert gas atmosphere to a suspension of K₂CO₃ (0.911 g, 6.6 mmol) and ethyl-1-benzyl-4-oxopiperidine-3-carboxylate (0.436 g, 1.65 mmol) in absolute THF (5 ml). The reaction mixture was refluxed for 17 h then filtered, and the filter residue was washed with THF (3×5 ml). The solvent was removed under vacuum and the residue purified by means of column chromatography (petroleum ether/ethyl acetate, 5:1, R_{f} 0.2). Yield: 0.563 g (72%),

¹H NMR (400 MHz, CDCl₃): δ=7.73 (s, 1H, ar.), 7.41 (d, J=8.1 Hz, 1H, ar.), 7.31 (d, J=8.1 Hz, 1H, ar.), 7.27-7.18 (m, 5H, N-benzyl ar.), 4.10-4.03 (m, 2H, CO₂CH₂CH₃), 3.56 (d, J=13.2 Hz, 1H, PhCH₂NR₂), 3.51-3.45 (m, 3H, PhCH₂NR₂, 2-H and ArCH₂), 3.11 (d, J=14.5 Hz, 1H, ArCH₂), 3.02-2.98 (m, 1H, 5-H), 2.94-2.85 (m, 1H, 5-H), 2.38 (d, J=14.5 Hz, 1H, 6-H), 2.29-2.26 (m, 2H, 2-H and 6-H), 1.07 (t, J=7.1, 3H, CO₂CH₂CH₃). ¹³C NMR (100 MHz, CDCl₃): δ=205.2 (C═O), 170.7 (CO₂Et), 142.9, 137.9 (2C quat. ar.), 136.2, 133.0, 130.8, 129.2, 128.7, 127.8 (6C tert. ar.), 126.6 (quat. ar.), 117.6 (CN), 112.4 (quat. ar.), 62.4, 62.1, 62.03, 61.50, 53.3, 40.8, 36.4 (7C sec.), 14.2 (CO₂CH₂CH₃).

### Ethyl-N-benzyl-9-cyano-11-oxo-1,3,4,6-tetrahydro-1,5-methano-3-benzazocine-5(2H)-carboxylate (16)

Absolute toluene (5 ml), K₃PO₄ (0.400 g, 1.88 mmol), Pd₂(dba)₂.CHCl₃ (13 mg, 2 mol %) and tBu₃P (0.7 ml of a 0.04 N solution in toluene, 4 mol %) were added under an inert gas atmosphere to ethyl-3-(2-bromo-4-cyanobenzyl)-N-benzyl-4-oxopiperidine-3-carboxylate (0.306 g, 0.64 mmol). The reaction mixture was stirred intensively for 15 h at 100° C. The reaction mixture was then cooled to room temperature, filtered off through Celite and washed with toluene (3×5 ml). The solvent was removed under vacuum and the residue purified by means of column chromatography (petroleum ether/ethyl acetate, 5:1, R_{f}=0.2). The product was obtained in a yield of 22% (0.055 g).

Melting point 158-160° C.

¹H NMR (400 MHz, CDCl₃): δ=7.78 (d, J=7.9 Hz, 1H, ar.), 7.53 (d, J=7.9 Hz, 1H, ar.), 7.46-7.39 (m, 5H, ar.), 7.10-7.08 (m, 2H, ar.), 4.47 (dd, J=14.4, 7.1 Hz, 2H, CO₂CH₂CH₃), 4.26 (d, J=18.5 Hz, 1H, 1-H), 3.83 (d, J=13.5 Hz, 1H, 6-H), 3.77 (d, J=13.5 Hz, 1H, 6-H), 3.70 (s, 2H, PhCH₂NR₂), 3.44 (dd, J=11.5, 1.5 Hz, 1H, 4-H), 3.38 (d, J=11.5 Hz, 1H, 4-H), 3.20 (ddd, J=10.9, 2.3 Hz, 1H, 2-H), 3.03 (dd, J=9.2, 2.0 Hz, 1H, 2-H), 1.51 (t, J=7.1 Hz, 3H, CO₂CH₂CH₃). ¹³C NMR (100 MHz, CDCl3): δ=203.9 (C═O), 168.0 (CO₂Et), 140.6 (quat. ar.), 129.7, 129.4, 127.0 (3C tert. ar.), 126.8, 125.9 (2C quat. ar.), 117.2 (CN), 108.8 (tert. ar.), 61.8, 60.5, 60.0, 58.9 (4C sec.), 57.4 (C-5 quat.), 50.6 (C-1 tert.), 39.6 (C-6 sec.) 12.71 (CO₂CH₂CH₃).

The structures of the example compounds are summarized in the following table:

| Compound no. | Structure | Name |
|---|---|---|
| 6 | 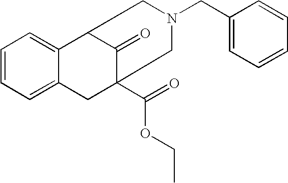 | Ethyl-N-benzyl-11-oxo-1,3,4,6-tetrahydro-1,5-methano-3-benzazocine-5(2H)-carboxylate |
| 7 | 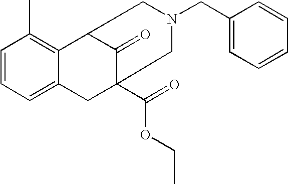 | Ethyl-N-benzyl-10-methyl-11-oxo-1,3,4,6-tetrahydro-1,5-methano-3-benzazocine-5(2H)-carboxylate |
| 8 | 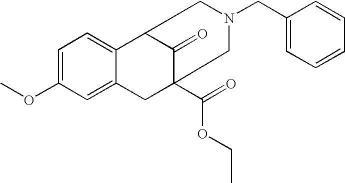 | Ethyl-N-benzyl-8-methoxy-11-oxo-1,3,4,6-tetrahydro-1,5-methano-3-benzazocine-5(2H)-carboxylate |
| 9 | 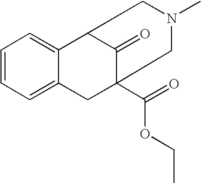 | Ethyl-N-methyl-11-oxo-1,3,4,6-tetrahydro-1,5-methano-3-benzazocine-5(2H)-carboxylatehydrochloride |
| 10 | 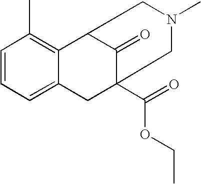 | Ethyl-N-10-dimethyl-11-oxo-1,3,4,6-tetrahydro-1,5-methano-3-benzazocine-5(2H)-carboxylate |
| 11 | 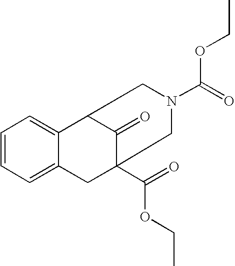 | Diethyl-11-oxo-1,3,4,6-tetrahydro-1,5-methano-3-benzazocine-3,5(2H)-dicarboxylate |
| 14 | 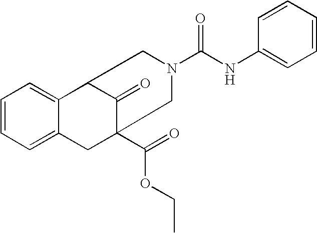 | Ethyl-N-(anilinocarbonyl)-11-oxo-1,3,4,6-tetrahydro-1,5-methano-3-benzazocine-5(2H)-carboxylate |
| 15 | 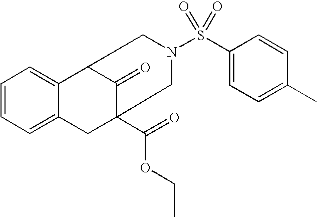 | Ethyl-N-tosyl-11-oxo-1,3,4,6-tetrahydro-1,5-methano-3-benzazocine-5(2H)-carboxylate |
| 16 | 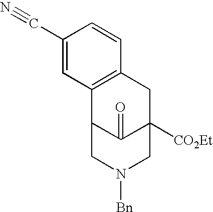 | Ethyl-N-benzyl-9-cyano-11-oxo-1,3,4,6-tetrahydro-1,5-methano-3-benzazocine-5(2H)-carboxylate |

Investigations into the Effectiveness of the Compounds According to the Invention

### Measurement of Serotonin Re-Uptake

Synaptosomes from rat brain areas were freshly isolated for the purposes of these in-vitro studies. In each case a “P₂” fraction was used, which was prepared in accordance with the instructions given by Gray and Whittaker (E. G. Gray und V. P. Whittaker (1962) J. Anat. 76, 79-88). For 5HT uptake these vesicular particles were isolated from the medulla and pons region of male rat brains.

A detailed description of the method can be found in the literature (M. Ch. Frink, H.-H. Hennies, W. Englberger, M. Haurand and B. Wilffert (1996) Arzneim.-Forsch./Drug Res. 46 (III), 11, 1029-1036).

### Measurement of Noradrenalin Re-Uptake

Synaptosomes from rat brain areas were freshly isolated for the purposes of these in-vitro studies. In each case a “P₂” fraction was used, which was prepared in accordance with the instructions given by Gray and Whittaker (E. G. Gray und V. P. Whittaker (1962) J. Anat. 76, 79-88). For NA uptake these vesicular particles were isolated from the hypothalamus of male rat brains.

A detailed description of the method can be found in the literature (M. Ch. Frink, H.-H. Hennies, W. Englberger, M. Haurand and B. Wilffert (1996) Arzneim.-Forsch./Drug Res. 46 (III), 11, 1029-1036).

The inhibition of serotonin and noradrenalin re-uptake was determined by way of example for compound 9:

| | 5-HT re-uptake | NA re-uptake |
|---|---|---|
| Compound | [10 μm], | [10 μm], |
| no. | % inhibition | % inhibition |
| 9 | 77 | 80 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A substituted tricyclic piperidone compound corresponding to Formula I: 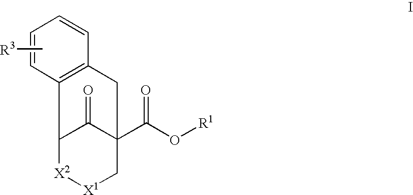
wherein
R¹ denotes methyl, ethyl or phenyl;
X₁ denotes NR² and X₂ denotes CH₂; or
X₂ denotes NR², and X₁ denotes CH₂;
R² denotes optionally mono- or polysubstituted, branched or unbranched, saturated or unsaturated C₁₋₈ alkyl optionally containing a heteroatom as a chain segment; optionally mono- or polysubstituted aryl, C₃₋₈ cycloalkyl or heteroaryl bonded via an optionally substituted, saturated or unsaturated, branched or unbranched C₁₋₈ alkylene group optionally containing a heteroatom as a chain segment; (C═O)R⁴; (C═O)NR⁵R⁶; (C═S)NR⁵R⁶; SO₂R⁷; or (C═O)OR⁸;
R³ denotes H; F; Cl; Br; OH; OCH₃; SCH₃; NO₂; CN; optionally mono- or polysubstituted, saturated or unsaturated, branched or unbranched C₁₋₈ alkyl or C₃₋₈ cycloalkyl; or phenyl, benzyl or phenethyl optionally mono- or polysubstituted with F, Cl, OH, OCH₃, SCH₃, NO₂, CN, CF₃, methyl, or ethyl;
R⁴, R⁷ and R⁸ each independently denote optionally mono- or polysubstituted, branched or unbranched, saturated or unsaturated C₁₋₈ alkyl or C₃₋₈ cycloalkyl optionally containing an N, O, or S heteroatom as a chain segment; optionally mono- or polysubstituted aryl or heteroaryl; or optionally mono- or polysubstituted aryl, C₃₋₈ cycloalkyl or heteroaryl bonded via a saturated or unsaturated, branched or unbranched, substituted or unsubstituted C₁₋₈ alkylene group optionally containing an N, O or S heteroatom as chain a segment;
R⁵ and R⁶ independently denote H; optionally mono- or polysubstituted, saturated or unsaturated, branched or unbranched C₁₋₈ alkyl or C₃₋₈ cycloalkyl optionally containing an N, O, S heteroatom as a chain segment; optionally mono or polysubstituted aryl or heteroaryl; or optionally mono- or polysubstituted aryl, C₃₋₈ cycloalkyl or heteroaryl bonded via an optionally substituted, saturated or unsaturated, branched or unbranched C₁₋₈ alkylene group, optionally containing an N, O or S heteroatom as a chain segment, with the proviso that R⁵ and R⁶ are not simultaneously H; or
R⁵ and R⁶ together form a saturated or unsaturated five-, six- or seven-membered non-aromatic ring optionally containing a further heteroatom selected from the group consisting of S, O or NR⁹ and optionally substituted with benzyl or C₁₋₅ alkyl;
wherein R⁹ denotes branched or unbranched C₁₋₅ alkyl, phenyl or benzyl, unsubstituted or mono- or polysubstituted with F, Cl, OH, OCH₃, SCH₃, NO₂, CN, CF₃, methyl or ethyl;
or a salt thereof with a physiologically compatible acid.

2. A compound as claimed in claim 1, wherein said compound is present in the form of an isolated stereoisomer.

3. A compound as claimed in claim 1, wherein said compound is present in the form of a mixture of stereoisomers.

4. A compound as claimed in claim 3, wherein said compound is present in the form of a racemic mixture.

5. A compound as claimed in claim 1, wherein X₁ denotes NR², and X₂ denotes CH₂.

6. A compound as claimed in claim 1, wherein R² denotes branched or unbranched, unsubstituted and saturated C₁₋₅ alkyl; or a phenyl, naphthyl, pyridyl, furyl, thienyl or indolyl group bonded via a C₁₋₃ alkylene group; wherein said C₁₋₅ alkyl or said phenyl, naphthyl, pyridyl, furyl, thienyl or indolyl group is optionally mono- or polysubstituted with —F, —Cl, —CF₃, —OCH₃, methyl, ethyl, n-propyl, nitro, tert-butyl or —CN.

7. A compound as claimed in claim 6, wherein R² denotes methyl or benzyl.

8. A compound as claimed in claim 1, wherein R² denotes (C═O)R⁴, (C═O)NR⁵R⁶, (C═S)NR⁵R⁶, SO₂R⁷ or (C═O)OR⁸.

9. A compound as claimed in claim 1, wherein R⁴, R⁷ and R⁸ each independently denote:
methyl, ethyl, n-propyl, isopropyl, n-butyl or tert-butyl;
phenyl, naphthyl, thienyl, furyl or pyridyl optionally mono- or polysubstituted with F, Cl, CF₃, NO₂, CH₃, OH, SH, OCH₃ or SCH₃; or
a phenyl, naphthyl, thienyl, furyl or pyridyl group bonded via a C₁₋₃ alkylene group and optionally mono- or polysubstituted with F, Cl, CF₃, NO₂, CH₃, OH, SH, OCH₃ or SCH₃.

10. A compound as claimed in claim 9, wherein R⁴, R⁷ and R⁸ each independently denote methyl, ethyl, phenyl or tosyl.

11. A compound as claimed in claim 1, wherein:
R⁵ and R⁶ each independently denote H; branched or unbranched C₁₋₅ alkyl or C₃₋₈ cycloalkyl; optionally mono- or polysubstituted aryl, or heteroaryl; or optionally mono- or polysubstituted aryl, C₃₋₈ cycloalkyl or heteroaryl bonded via a C₁₋₃ alkylene group: or
R⁵ and R⁶ together form a piperidine, piperazine, morpholine or thiomorpholine ring.

12. A compound as claimed in claim 11, wherein R⁵ and R⁶ independently denote
H;
methyl, ethyl, n-propyl, isopropyl, n-butyl or tert-butyl;
phenyl, naphthyl, thienyl, furyl or pyridyl optionally mono- or polysubstituted with F, Cl, CF₃, NO₂, CH₃, OH, SH, OCH₃ or SCH₃; or
phenyl, naphthyl, thienyl, furyl or pyridyl bonded via a C₁₋₃ alkylene group and optionally mono- or polysubstituted with F, Cl, CF₃, NO₂, CH₃, OH, SH, OCH₃ or SCH₃.

13. A compound as claimed in claim 12, wherein R⁵ and R⁶ each independently denote H or phenyl.

14. A compound as claimed in claim 1, wherein R³ denotes H, F, Cl, OH, OCH₃, SCH₃, NO₂, CN, CF₃, methyl, ethyl or benzyl.

15. A compound as claimed in claim 14, wherein R³ denotes H, CN, OCH₃ or methyl.

16. A compound as claimed in claim 1, selected from the group consisting of:
ethyl-N-benzyl-11-oxo-1,3,4,6-tetrahydro-1,5-methano-3-benzazocine-5(2H)-carboxylate;
ethyl-N-benzyl-10-methyl-11-oxo-1,3,4,6-tetrahydro-1,5-methano-3-benzazocine-5(2H)-carboxylate;
ethyl-N-benzyl-8-methoxy-11-oxo-1,3,4,6-tetrahydro-1,5-methano-3-benzazocine-5(2H)-carboxylate;
ethyl-N-methyl-11-oxo-1,3,4,6-tetrahydro-1,5-methano-3-benzazocine-5(2H)-carboxylate hydrochloride;
ethyl-N-10-dimethyl-11-oxo-1,3,4,6-tetrahydro-1,5-methano-3-benzazocine-5(2H)-carboxylate;
diethyl-11-oxo-1,3,4,6-tetrahydro-1,5-methano-3-benzazocine-3,5(2H)-dicarboxylate
ethyl-N-(anilinocarbonyl)-11-oxo-1,3,4,6-tetrahydro-1,5-methano-3-benzazocine-5(2H)-carboxylate;
ethyl-N-tosyl-11-oxo-1,3,4,6-tetrahydro-1,5-methano-3-benzazocine-5(2H)-carboxylate, and
ethyl-N-benzyl-9-cyano-11-oxo-1,3,4,6-tetrahydro-1,5-methano-3-benzazocine-5(2H)-carboxylate;
or a salt thereof with a physiologically compatible acid.

17. A pharmaceutical composition comprising a compound as claimed in claim 1 and at least one pharmaceutically acceptable carrier or auxiliary substance.

18. A method for producing a substituted tricyclic piperidone compound as claimed in claim 1, said method comprising:
reacting a compound corresponding to formula B with a benzyl bromide in an organic solvent in the presence of a base at a temperature of 0 to 100° C. to form a compound corresponding to formula C, 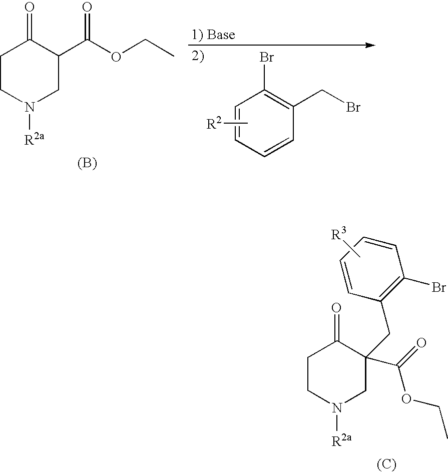
reacting the compound of formula C with a catalyst and a corresponding ligand in an organic solvent in the presence of a base, at a temperature of 40 to 130° C. under inert gas to form a compound corresponding to the formula Ia, 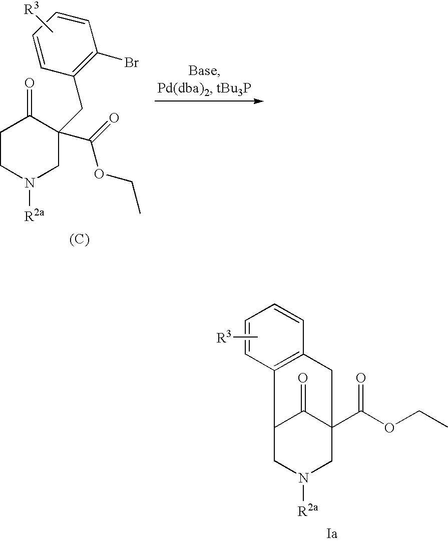
optionally dissolving the compound of formula Ia in a compound corresponding to the formula CIC(O)OR⁸, optionally in the presence of an additional solvent, and reacting the compound of formula Ia and the compound of formula CIC(O)OR⁸ at a temperature of 0 to 100° C. to form a compound corresponding to the formula Ib, 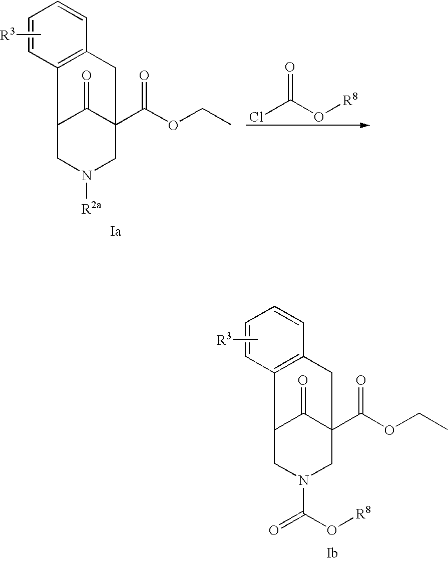
and
optionally reacting the compound of formula Ib in an organic solvent with trimethylsilyliodide at a temperature of −20 to 100° C. and then at a temperature of −20 to 100° C. with an electrophile selected from the group consisting of acid chlorides, acid anhydrides, sulfonic acid chlorides, isocyanates and isothiocyanates in an organic solvent in the presence of a base to form a compound corresponding to formula Ic, 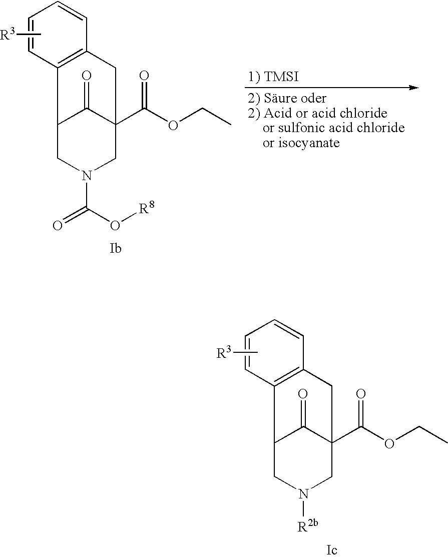
wherein
R^{2a} denotes optionally mono- or polysubstituted, saturated or unsaturated, branched or unbranched C₁₋₈ alkyl optionally containing a heteroatom as chain a segment; or an optionally mono- or polysubstituted aryl, C₃₋₈ cycloalkyl or heteroaryl group bonded via an optionally substituted, saturated or unsaturated, branched or unbranched C₁₋₈ alkyl group optionally containing a heteroatom as a chain segment; and
R^{2b} denotes (C═O)R⁴, (C═O)NR⁵R⁶, (C═S)NR⁵R⁶, SO₂R⁷ or (C═O)OR⁸.

19. A method as claimed in claim 18, wherein said catalyst is Pd(dba)₂, PdCl₂, or Pd(OAc)₂, and said ligand is t-Bu₃P or Ph₃P; or wherein said compound corresponding to the formula CIC(O)OR⁸ is benzyloxycarbonylchloride.

20. A method for producing a substituted tricyclic piperidone compound as claimed in claim 1, said method comprising:
reacting a compound corresponding to formula H with a benzyl bromide in an organic solvent in the presence of a base at a temperature of 0 to 100° C. to form a compound corresponding to formula I: 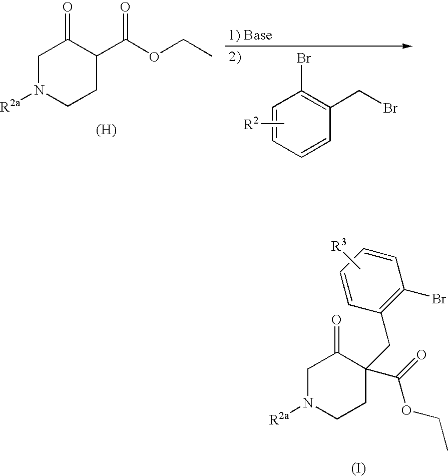
reacting the compound of formula I with a catalyst and a corresponding ligand in an organic solvent in the presence of a base at a temperature of 40 to 130° C. under inert gas to form a compound corresponding to formula Id, 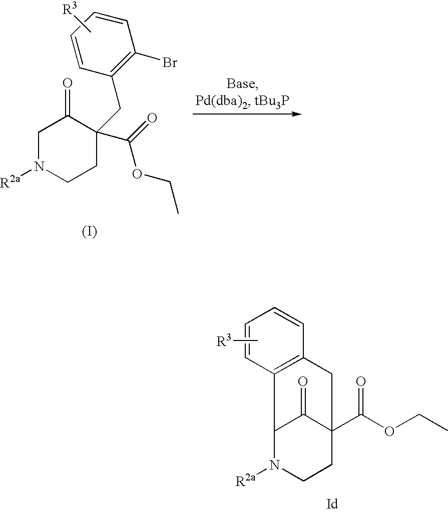
optionally dissolving the compound of formula Id in a compound corresponding to the formula CIC(O)OR⁸, optionally in the presence of an additional solvent, and reacting the compound of formula Id and the compound of the formula CIC(O)OR⁸ at a temperature of 0 to 100° C. to form a compound corresponding to formula Ie, 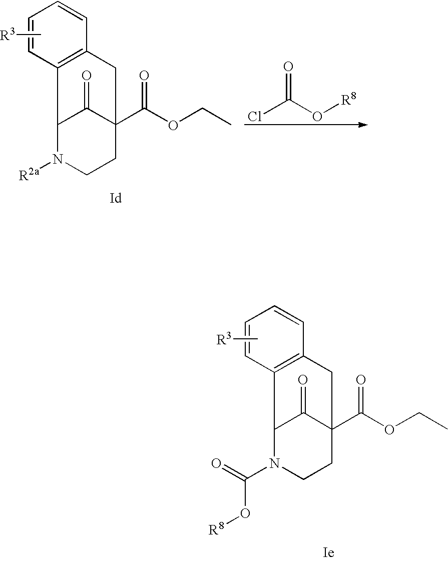
and
optionally reacting the product corresponding to formula Ie in an organic solvent with trimethylsilyliodide at a temperature of −20 to 100° C. and then at a temperature of −20 to 100° C. with an electrophile selected from the group consisting of acid chlorides, acid anhydrides, sulfonic acid chlorides, isocyanates and isothiocyanates, in an organic solvent in the presence of a base to form a compound corresponding to formula If, 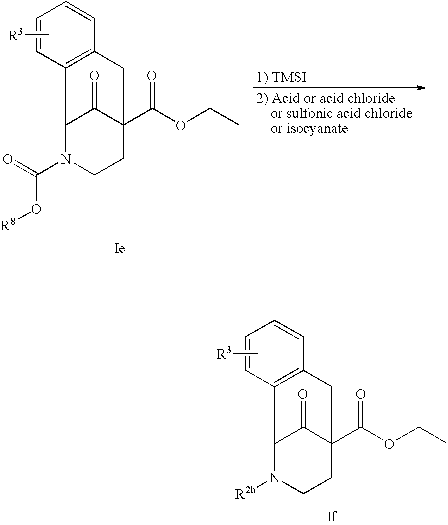
wherein
R^{2a} denotes optionally mono- or polysubstituted, saturated or unsaturated, branched or unbranched C₁₋₈ alkyl optionally containing a heteroatom as a chain segment; or optionally mono- or polysubstituted aryl, C₃₋₈ cycloalkyl or heteroaryl bonded via an optionally substituted, saturated or unsaturated, branched or unbranched C₁₋₈ alkylene group optionally containing a heteroatom as a chain segment; and
R^{2b} denotes (C═O)R⁴, (C═O)NR⁵R⁶, (C═S)NR⁵R⁶, SO₂R⁷ or (C═O)OR⁸.

21. A method as claimed in claim 20, wherein said catalyst is Pd(dba)₂, PdCl₂, or Pd(OAc)₂, and said ligand is t-Bu₃P or Ph₃P; or wherein said compound corresponding to the formula CIC(O)OR⁸ is benzyloxycarbonylchloride.

22. A method of treating a condition selected from the group consisting of pain, depression, urinary incontinence, diarrhea, pruritus, alcohol and drug abuse, drug dependency, lethargy and anxiety in a subject in need thereof, said method comprising administering to said subject a pharmacologically effective amount of a compound as claimed in claim 1.

23. A method as claimed in claim 22, wherein said condition is pain.

24. A method as claimed in claim 23, wherein said condition is acute pain, neuropathic pain or chronic pain.
